(19) United States
(12) Patent Application Publication
(10) Pub. No.: US 2025/0268459 A1
(43) Pub. Date: Aug. 28, 2025

(54) ENDOSCOPE FIXING STRUCTURE

(71) Applicant: SHANGHONG MEDICAL EQUIPMENT COMPANY LIMITED, New Taipei City (TW)

(72) Inventors: CHING-HAO YU, New Taipei City (TW); CHIA-RONG SU, New Taipei City (TW); CHENG-TE LO, New Taipei City (TW)

(21) Appl. No.: 18/590,899

(22) Filed: Feb. 28, 2024

(51) Int. Cl. A61B 1/00; A61B 1/233; A61J 15/00

(52) U.S. Cl. CPC A61B 1/00148; A61B 1/00154; A61B 1/233; A61J 15/0003

(57) ABSTRACT

An endoscope fixing structure mainly includes an endoscope, an image pickup assembly, and a fixing member. The endoscope is extendable into a nasogastric tube. The image pickup assembly is arranged at an end of the endoscope. The fixing member is arranged at one side of the endoscope that is opposite to the image pickup assembly. The fixing member includes a front end portion and a rear end portion having a cross-sectional area greater than the front end portion and is thus fixable to an opening of the nasogastric tube by means of the rear end portion to constrain movement of the endoscope. To combine the endoscope with the nasogastric tube, the image pickup assembly is first inserted into the nasogastric tube, until retaining engagement is induced between the fixing member and the opening of the nasogastric tube. This achieves fast fixing of the endoscope to the nasogastric tube.

### BACKGROUND OF THE INVENTION

### (a) Technical Field of the Invention

The present invention relates to an endoscope fixing structure for fast fixing an endoscope to a nasogastric tube.

### (b) Description of the Prior Art

To simultaneously insert a nasogastric tube and an endoscope, due to the endoscope and the nasogastric tube being not fixed together, the two cannot be conducted synchronously during the course of insertion, making them obstructive in the course of insertion. In view of this, manufacturers developed a technique that allows the nasogastric tube and the endoscope to be fixed together in order to reduce obstructiveness thereof. An example is Taiwan Utility Model M637407, which discloses a system for assisting disposition of nasogastric/nasoenteric tube, in which a three-passage connector has a first passage that is set in communication and connection with a tube for disposition, and a second passage receive an endoscope device to insert therein so that the a channel image of a human body insertion portion can be acquired with the endoscope device to allow for operation and control of the disposed tube into a target channel and position, meanwhile, gas can be introduced through a third passage to allow the gas to expand the internal chamber of the human body to enhance clearness of the channel image of the interior of the human body and thus reduce the error rate of intubation and help enhance operation safety.

Although the above-described system for assisting disposition of nasogastric/nasoenteric tube, when used, is effective in fixing a nasogastric tube and an endoscope together, yet it can only be implemented with a three-passage connector. In other words, an endoscope cannot be fixed with a nasogastric tube without applying a third-party technology.

Other manufacturers proposed arranging a fixing structure for an endoscope and a nasogastric tube at a location adjacent to an end of a camera assembly, meaning fixing can only be achieved by having the fixing structure moved with the camera assembly of the endoscope to get into the nasogastric tube. This measure is also effective in achieving fixing, yet the space of the interior of the nasogastric tube is very limited, so that arranging the fixing structure at the end of the camera assembly can only be implemented by adopting structure miniaturization technology, and this causes a significant increase of cost.

### SUMMARY OF THE INVENTION

The primary objective of the present invention is to use an arrangement that a fixing member has a front end portion and a relatively wide rear end portion to allow for fast fitting engagement and thus fixing of an endoscope to a nasogastric tube, without application of a third-party component, and exhibiting advantages of simple structure and time and cost saving.

A main structure of the present invention for achieving the above primary objective comprises an endoscope, which is extendable into a nasogastric tube, the endoscope being provided, at an end thereof, with an image pickup assembly, and a fixing member at an opposite end, wherein the fixing member comprises a front end portion and a rear end portion having a cross-sectional area greater than the front end portion formed through extending from the front end portion.

For an attempt to fast fixing the endoscope to the nasogastric tube, after the image pickup assembly is disposed into the nasogastric tube, a user may make the fixing member of the endoscope to form a tight fitting engagement with respect to an opening of the nasogastric tube, meaning retaining engagement is formed, and the retaining engagement is fitting engagement between the rear end portion and a wall of the nasogastric tube to allow for fast achieving fixing of the endoscope to the nasogastric tube.

By means of the above techniques, the drawback of the prior art system for assisting disposition of nasogastric/nasoenteric tube that although it is effective to fix the nasogastric tube and the endoscope together, yet this is only implementable with a three-passage connector and this means the endoscope cannot be fixed with the nasogastric tube without applying a third-party technology can be overcome to achieve utilization of the present invention for the above-described advantages.

Another objective of the present invention is that the present invention can be made in an externally mountable configuration having a combining portion that is applicable to combine the fixing member with an endoscope that originally does not include a fixing member to provide extremely wide applications.

A main structure of the present invention for achieving said another objective comprises a fixing member arranged on an endoscope that is extendable into a nasogastric tub, wherein the fixing member comprises a front end portion and a rear end portion having a cross-sectional area greater than the front end portion, so as to be fixable to an opening of the nasogastric tube to constrain movement of the endoscope, and the fixing member is provided with a combining portion arranged thereon to receive the endoscope to attach thereto or detach therefrom.

For an endoscope long existing and available in the market to be mounted to a nasogastric tube, the combining portion is applied for fixing with the endoscope, and under this condition, the endoscope becomes including a fixing member, and during penetration into the nasogastric tube, the rear end portion of the fixing member is brought into contact with the opening of the nasogastric tube for fixing to make the endoscope not possible for further moving thereby achieving an effect of fixing and positioning.

The problem of the prior art technology that although it is also effective for fixing, the interior space of the nasogastric tube is limited and arranging a structure for fixing on an end of an image pickup assembly is only implementable with a miniaturization structure manufacturing process that causes a significant increase of the manufacturing cost can be overcome with the present invention to achieve utilization of the present invention for the above-described advantages.

### BRIEF DESCRIPTION OF THE DRAWINGS

FIG. 1 is an exploded view, partly see-through, showing a first embodiment of the present invention.

FIG. 2 is a schematic view showing disposition of an endoscope through insertion according to the first embodiment of the present invention.

FIG. 3 is a schematic view showing disposition of an endoscope through insertion according to a second embodiment of the present invention.

FIG. 4 is a schematic view showing an inclined portion of a third embodiment of the present invention.

FIG. 5 is a schematic view showing a signal transmission element of a fourth embodiment of the present invention.

FIG. 6 is a schematic view showing a display device of a fifth embodiment of the present invention.

FIG. 7 is a side elevational view showing a sixth embodiment of the present invention.

FIG. 8 is a side elevational view showing a seventh embodiment of the present invention.

FIG. 9 is a perspective view showing an eighth embodiment of the present invention.

FIG. 10 is a cross-sectional view, taken along line A-A of FIG. 9, showing the eighth embodiment of the present invention.

FIG. 11 is a first schematic view showing combination of the eighth embodiment of the present invention.

FIG. 12 is a second schematic view showing combination of the eighth embodiment of the present invention.

FIG. 13 is a perspective view showing a ninth embodiment of the present invention.

FIG. 14 is a perspective view showing a tenth embodiment of the present invention.

FIG. 15 is a perspective view showing an eleventh embodiment of the present invention.

FIG. 16 is a perspective view showing a twelfth embodiment of the present invention.

FIG. 17 is a perspective view showing a thirteenth embodiment of the present invention.

### DETAILED DESCRIPTION OF THE PREFERRED EMBODIMENTS

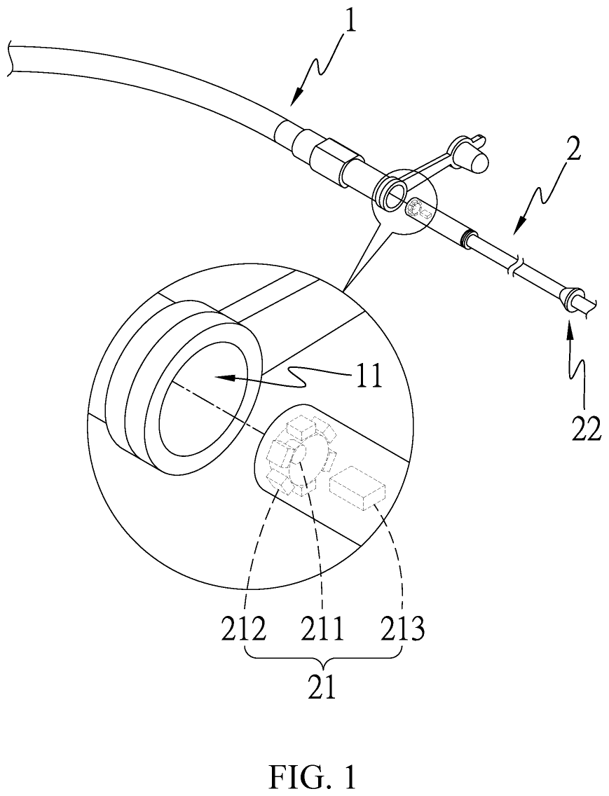

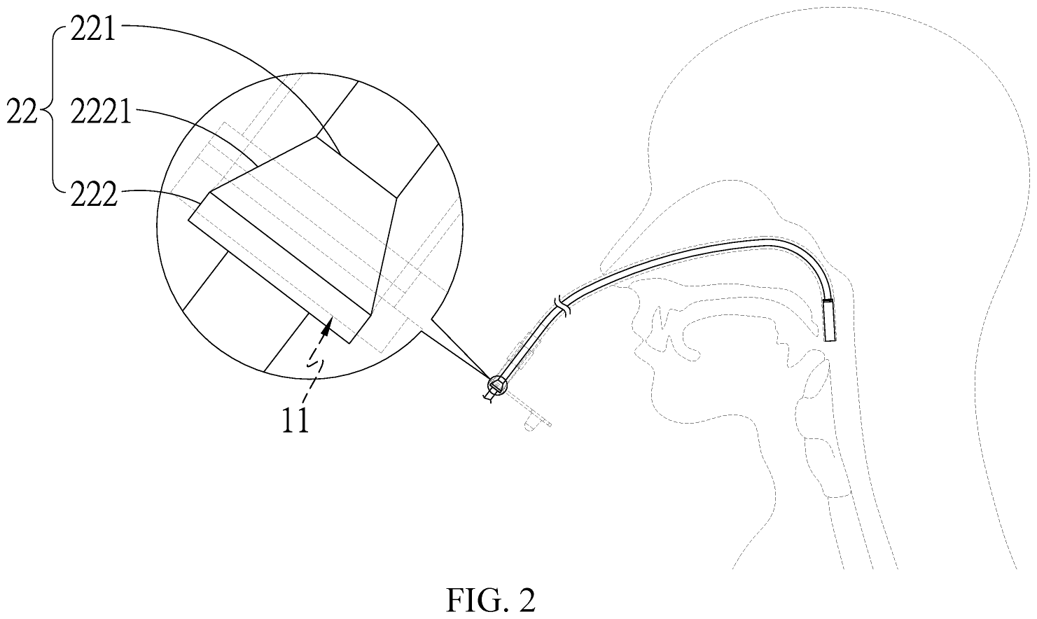

Referring to FIGS. 1 and 2, the present invention comprises: an endoscope 2, which is extendable into and through a nasogastric tube 1; an image pickup assembly 21, which is arranged at an end of the endoscope 2; and a fixing member 22, which is arranged at one side of the endoscope 2 that is opposite to the image pickup assembly 21, the fixing member 22 comprising a front end portion 221, a rear end portion 222 having a cross-sectional area greater than the front end portion 221, and an inclined portion 2221 located between the front end portion 221 and the rear end portion 222, wherein the fixing member 22 is fixable by the rear end portion 222 to an opening 11 of the nasogastric tube 1 in order to constrain movement of the endoscope 2.

A cross-sectional area of the front end portion 221 is smaller than a cross-sectional area of the opening 11 of the nasogastric tube 1.

The image pickup assembly 21 includes an image pickup lens 211, at least one light-emitting element 212 arranged at one side of the image pickup lens 211, and a signal transmission element 213 arranged at one side of the image pickup lens 211.

The light-emitting element 212 may be a light-emitting diode (LED).

Wireless transmission is taken as an example for illustrating the signal transmission element 213 in the instant embodiment.

When a user attempts to dispose the nasogastric tube 1 into the nasal cavity of a patient, to prevent the nasogastric tube 1 from being mistakenly inserted into respiratory tract, the endoscope 2 is applied as a measure for prevention by lighting up the interior of the human body with the light-emitting element 212 of the image pickup assembly 21 to allow the image pickup lens 211 to clearly pick up an instantaneous image for observation by the user, and the photograph image can be transmitted wirelessly with the signal transmission element 213. More importantly, in mounting the endoscope 2 in the interior of the nasogastric tube 1, the front end portion 221 of the fixing member 22 is moved into the interior of the nasogastric tube 1 first, and then, the inclined portion 2221 is subsequently moved into the interior of the nasogastric tube 1, and at this moment, due to the cross-sectional area of the rear end portion 222 being made equal to the cross-sectional area of the opening 11, friction is induced between the rear end portion 222 and the opening 11 to form tight fitting engagement for fixing. As such, the present invention uses the arrangement of the fixing member 22 to fast fulfill fixing and combining of the endoscope 2 with the nasogastric tube 1 by means of a simple structure.

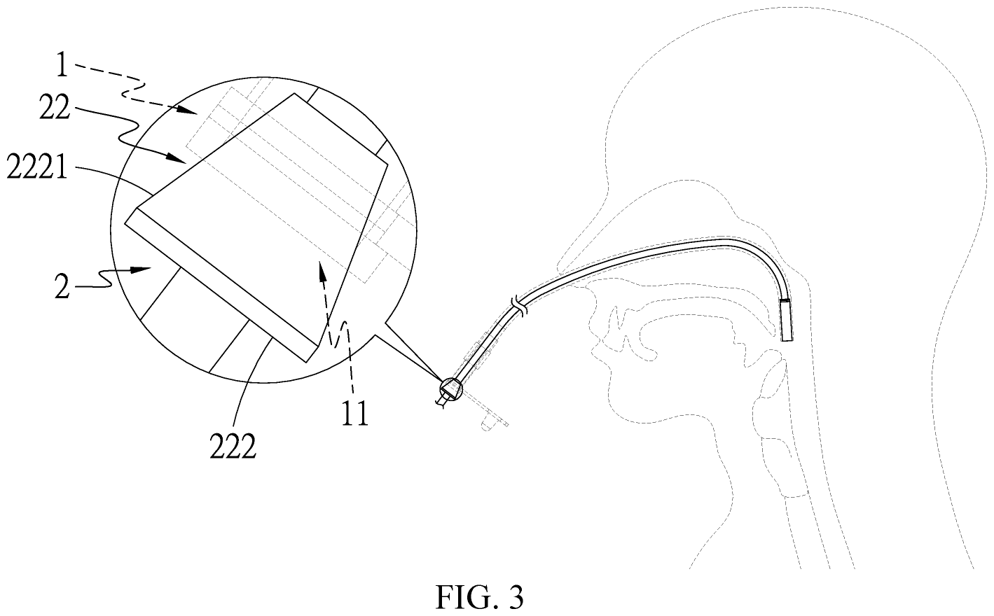

Referring to FIG. 3, in the instant embodiment, the cross-sectional area of the rear end portion 222 is greater than the cross-sectional area of the opening 11 of the nasogastric tube 1. When the endoscope 2 is being placed into the nasogastric tube 1, the inclined portion 2221 of the fixing member 22 is brought into contact with a circumference of the opening 11 of the nasogastric tube 1, so that when the user further applies an inward-directing force, the inclined portion 2221 forces a wall of the nasogastric tube 1 to expand outward to thereby fulfill tight fitting and fixing with the inclined portion 2221.

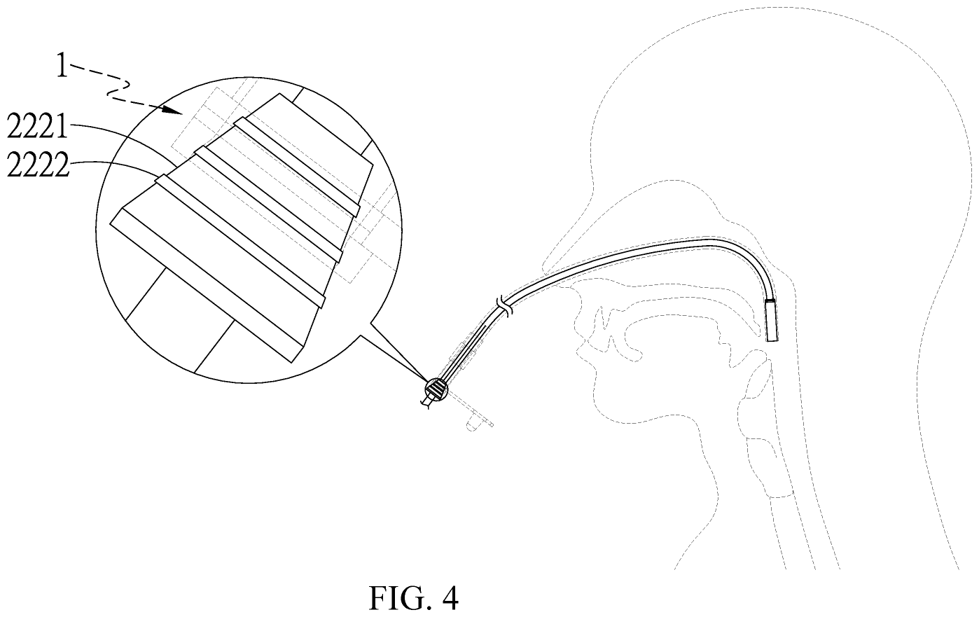

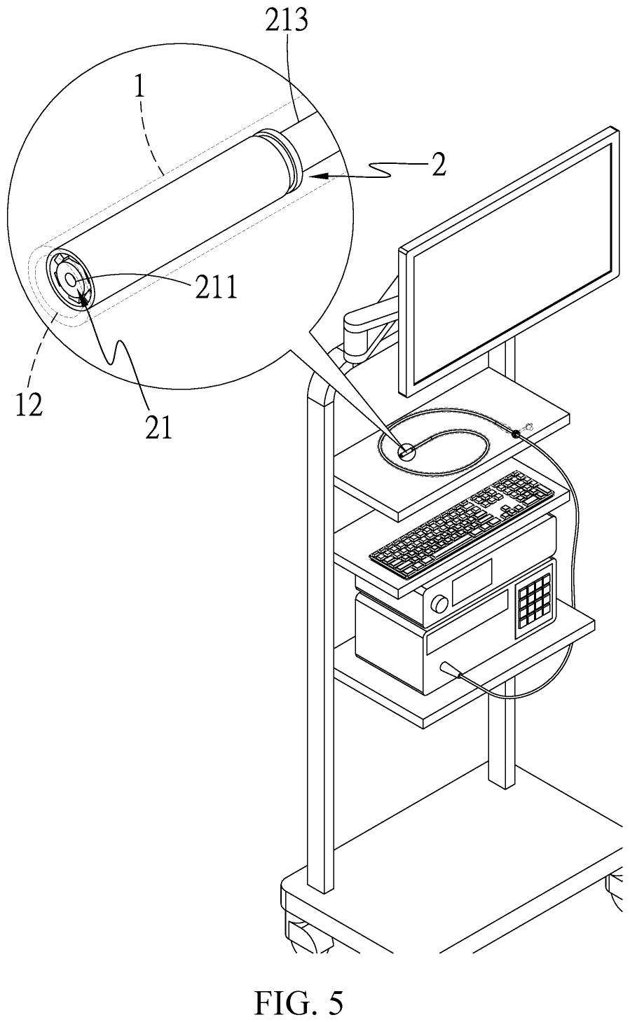

Referring to FIG. 4, in the instant embodiment, the inclined portion 2221 includes at least the rib 2222. In other words, by means of the arrangement of the rib 2222, the wall of the nasogastric tube 1 can be expanded further to fulfill a better effect of fixing. Referring to FIG. 5, in the instant embodiment, wired transmission is taken as an example to illustrate the signal transmission element 213. This indicates that in addition to wireless transmission, the present invention may alternatively or additionally use a wired transmission solution to transmit an image picked up by the image pickup lens 211, achieving an advantage of diversification. Further, to allow the image pickup assembly 21 that penetrates into the interior of the nasogastric tube 1 to pick up an image outside of the nasogastric tube 1, namely that of the windpipe or the food pipe, the nasogastric tube 1 is provided, at one end, with an end opening 12, so that the image pickup assembly 21 of the endoscope 2 is allowed to pick up an outside appearance image of the windpipe or the food pipe through the end opening 12, and then, the signal transmission element 213 may transmit the image to be observed by the user. In this way, with the open, unshielded arrangement of the end opening 12, the image pickup assembly 21 is allowed to conduct, in a clearer way, pickup of images at locations in front of the nasogastric tube 1 in a moving route in order to effectively reduce the chance of error decision. Further, generally, an operation of combining the nasogastric tube 1 and the endoscope 2 is only performable by medical personnel, and non-medical professional persons are not capable of or do not dare to use such devices. The present invention proposes the end opening 12 formed at one end of the nasogastric tube 1 allows the medical personnel to use the end opening 12 to allow the image pickup assembly 21 pick up outside appearance images of the windpipe or the food pipe, allowing the nasogastric tube 1 to accurately move into the food pipe.

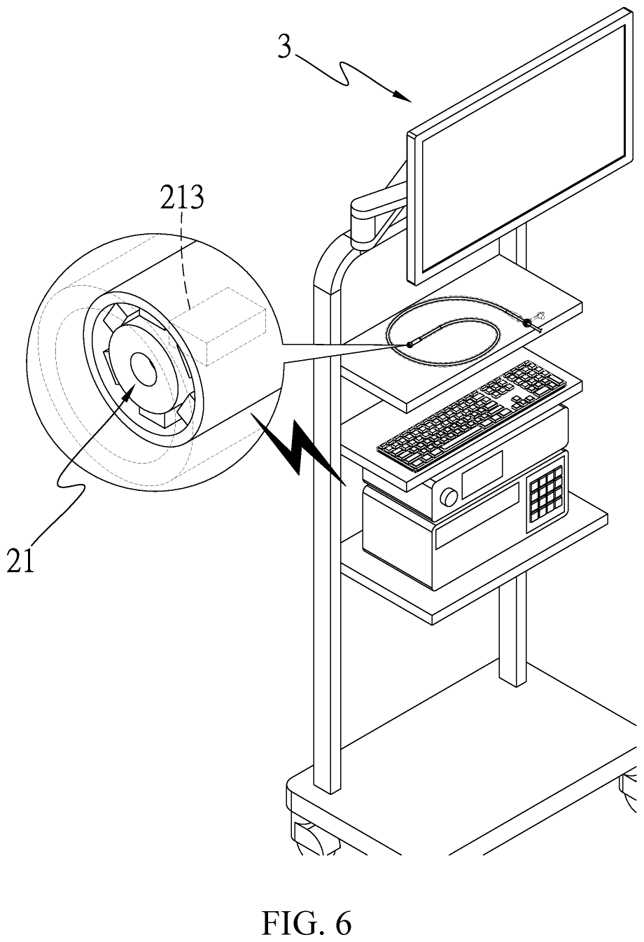

Referring to FIG. 6, in the instant embodiment, the signal transmission element 213 uses a wireless manner to establish information connection with a display device 3, and the signal transmission element 213 allows the image picked up by the image pickup assembly 21 to be transmitted to the display device 3 for instantaneous observation by the user.

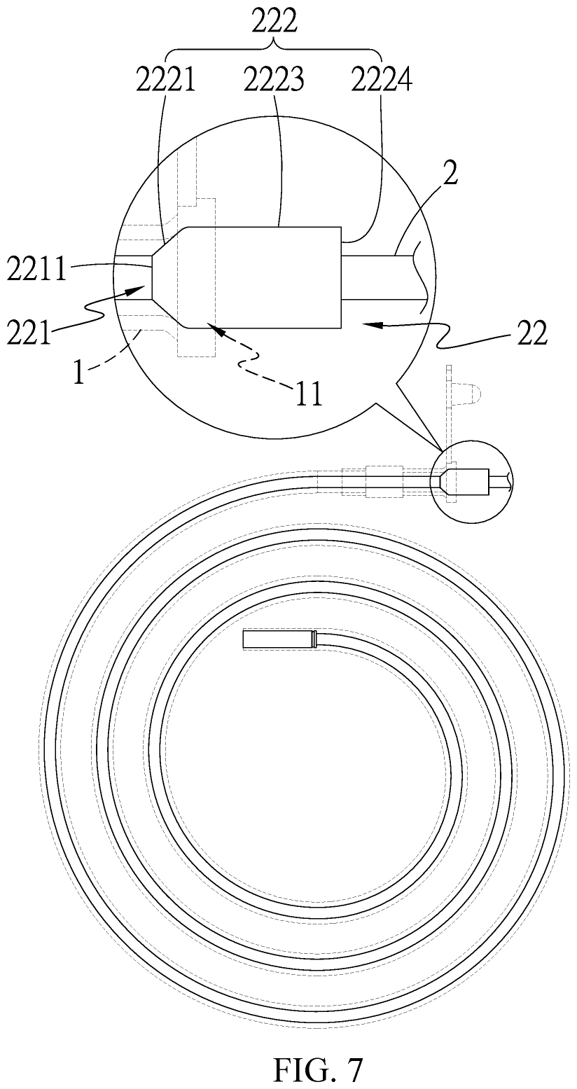

Referring to FIG. 7, in the instant embodiment, the overall configuration of the fixing member 22 is made different. In the instant embodiment, the front end portion 221 of the fixing member 22 includes a first planar portion 2211, while the rear end portion 222 comprises an inclined portion 2221, and a straight portion 2223 arranged at a rear end of the inclined portion 2221, and a second planar portion 2224 is arranged at one end of the straight portion 2223 that is opposite to the inclined portion 2221. As such, when the endoscope 2 is being inserted into the nasogastric tube 1 and reaches a predetermined depth, the inclined portion 2221 gets into mutual contact with the opening 11 of the nasogastric tube 1 to fix thereto thereby fulfilling an effect of fixing.

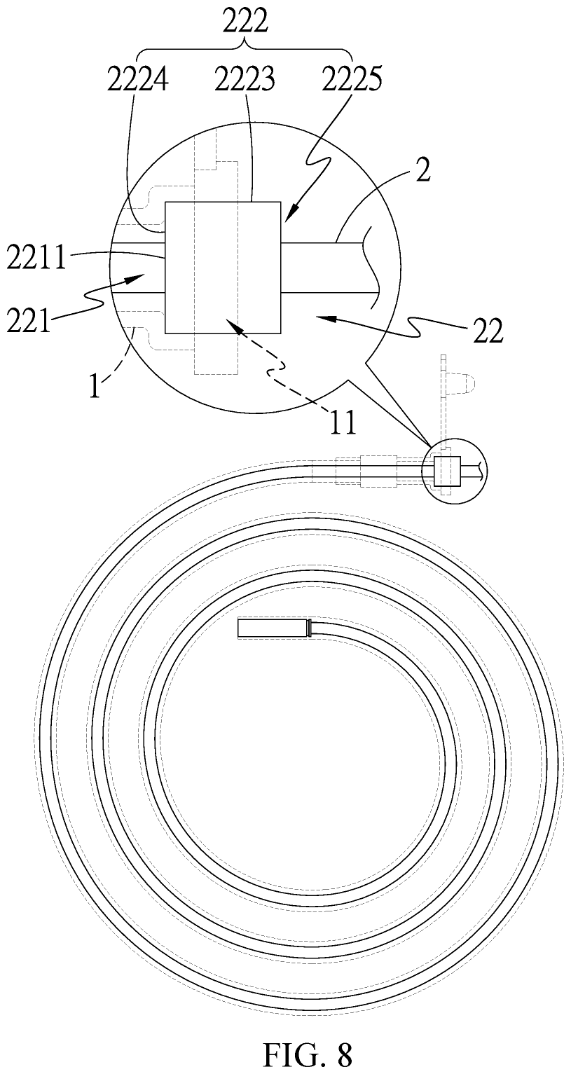

Referring to FIG. 8, in the instant embodiment, the overall configuration of the fixing member 22 is made different. In the instant embodiment, the front end portion 221 of the fixing member 22 includes a first planar portion 2211, while the rear end portion 222 comprises a second planar portion 2224, and a straight portion 2223 is extended rearwards from the second planar portion 2224, and a third planar portion 2225 is formed through further extending from the straight portion 2223. As such, when the endoscope 2 is being inserted into the nasogastric tube 1 and reaches a predetermined depth, the second planar portion 2224 gets into mutual contact with the opening 11 of the nasogastric tube 1 to fix thereto thereby fulfilling an effect of fixing.

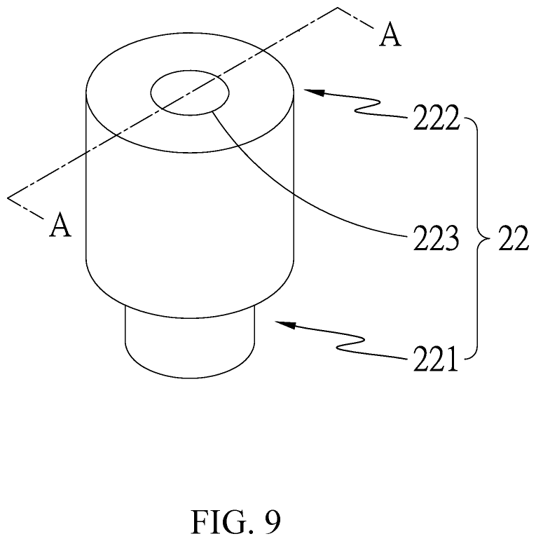

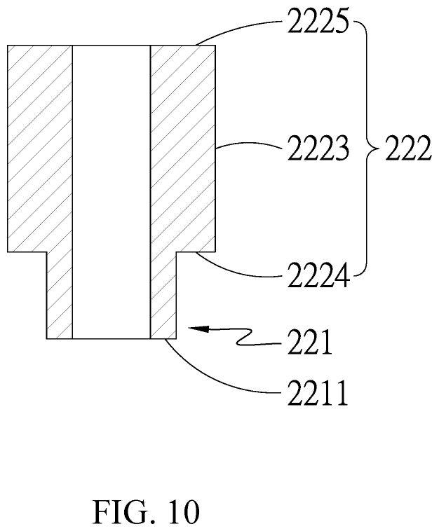

Referring to FIGS. 9 and 10, the invention mainly comprises:

a fixing member 22, which is arranged on an endoscope that is extendable into and through a nasogastric tube, the fixing member 22 comprising a front end portion 221 and a rear end portion 222 having a cross-sectional area greater than the front end portion 221 to be fixable to an opening of the nasogastric tube in order to constrain movement of the endoscope; and a combining portion 223, which is arranged on the fixing member 22 to receive the endoscope to attach thereto or detach therefrom.

A cross-sectional area of the front end portion 221 is smaller than a cross-sectional area of the opening of the nasogastric tube.

The cross-sectional area of the rear end portion 222 is greater than the cross-sectional area of the opening.

In the instant embodiment, the fixing member 22 is an externally mounted fixing member 22, and the front end portion 221 of the fixing member 22 includes a first planar portion 2211, while the rear end portion 222 comprises a second planar portion 2224, and a straight portion 2223 is extended rearwards from the second planar portion 2224, and a third planar portion 2225 is formed through extending from the straight portion 2223.

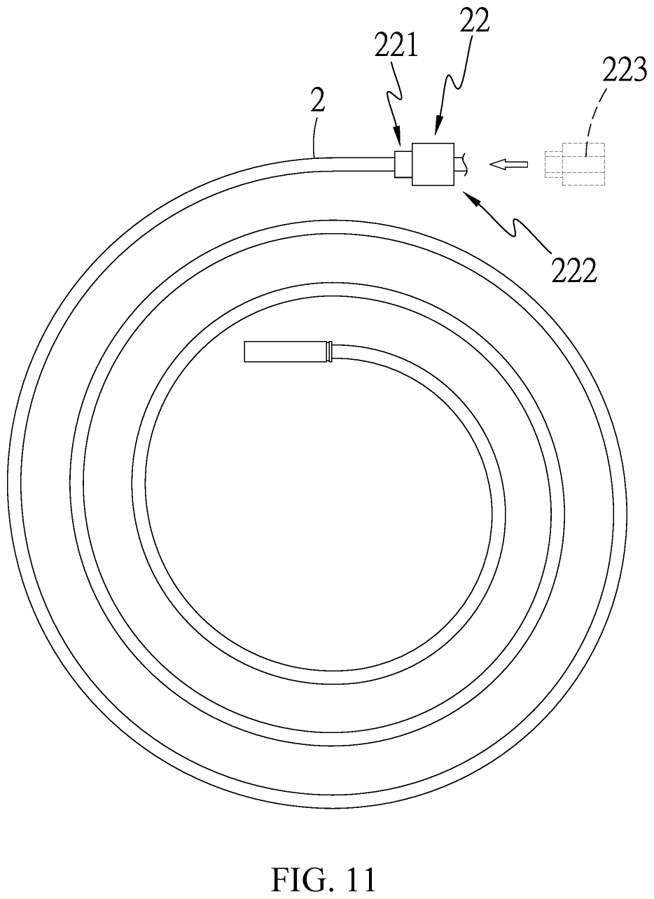

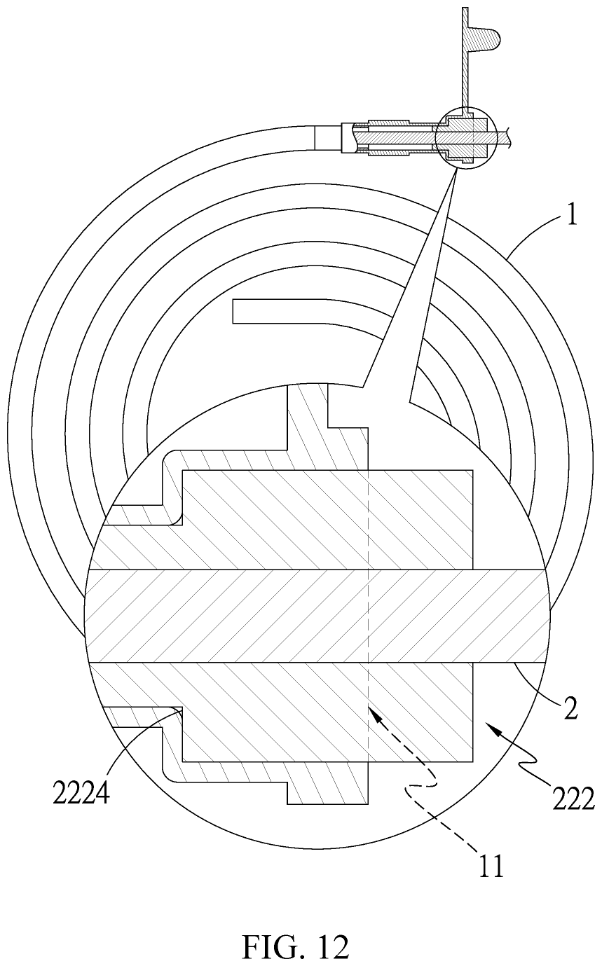

Referring to FIGS. 9-12, in the instant embodiment, a through hole is taken as an example to illustrate the combining portion 223, and is combinable with the endoscope 2 through fitting in a front-rear direction, so that when the user attempts to dispose the nasogastric tube 1 into the nasal cavity of a patient, the fixing member 22 is first fit to and thus combined with the endoscope 2 by means of the combining portion 223, and while the endoscope 2 is penetrating through the nasogastric tube 1, the front end portion 221 of the fixing member 22 is moved into the interior of the nasogastric tube 1 first, and then, due to the cross-sectional area of the rear end portion 222 being greater than the cross-sectional area of the opening 11, the second planar portion 2224 is brought into abutting engagement and thus contacting with a wall of the opening 11 to form tight engagement for fixing. As such, the present invention uses the arrangement of the fixing member 22 to fast fulfill fixing and combining of the endoscope 2 with the nasogastric tube 1 by means of a simple structure. This means the fixing member 22 of the instant embodiment is an externally mounted device, so that when an existing endoscope 2 does not have a function of mutually abutting and fixing with the nasogastric tube 1, the fixing member 22 of the present invention can be externally or additionally mounted on the endoscope 2, so that the endoscope 2 that originally does not include an effect of fixing is turned into an endoscope 2 that includes an effect of fixing. As a whole, an effect of fixing can be fulfilled between the endoscope 2 and the nasogastric tube 1 with the lowest cost.

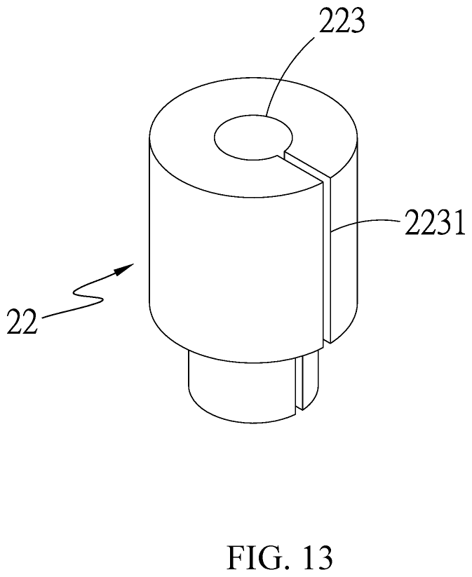

Referring to FIG. 13, in the instant embodiment, the combining portion 223 is formed, in one side thereof, with a groove 2231, and the groove 2231 is in communication with the combining portion 223. The groove 2231 includes a deformable elastic material, so that when it is attempted to combine the fixing member 22 with an endoscope, the endoscope may be operated to penetrate through the groove 2231 to combine with and held in the combining portion 223, the combination being made in an up-down fashion. This suggests that the combination between the fixing member 22 and an endoscope according to the present invention is not limited to any specific way.

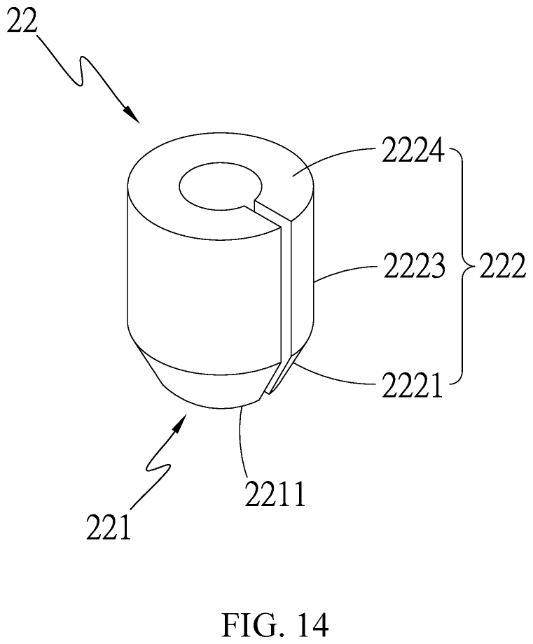

Referring to FIG. 14, in the instant embodiment, an externally mountable fixing member 22 is provided, showing a different overall configuration. In the instant embodiment, the front end portion 221 of the fixing member 22 includes a first planar portion 2211, while the rear end portion 222 comprises an inclined portion 2221, and a straight portion 2223 is arranged at a rear end of the inclined portion 2221, and a second planar portion 2224 is arranged at one end of the straight portion 2223 that is opposite to the inclined portion 2221. As such, the endoscope is being inserted into the nasogastric tube and reaches a predetermined depth, the inclined portion gets into mutual contact with the opening of the nasogastric tube to fix thereto thereby fulfilling an effect of fixing.

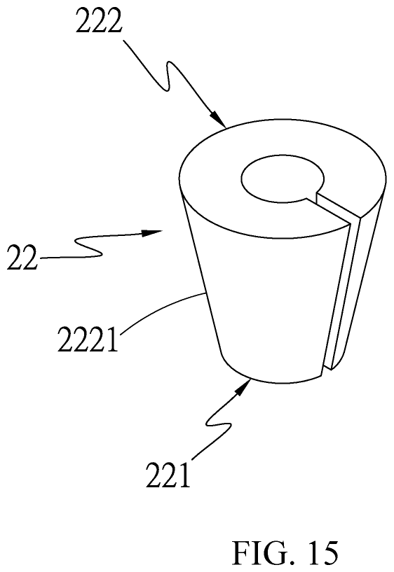

Referring to FIG. 15, in the instant embodiment, a detachable fixing member 22 is provided, and a conic form is taken as an example for illustration, wherein an inclined portion 2221 is arranged between the front end portion 221 and the rear end portion 222 to make the cross-sectional area of the rear end portion 222 greater than the cross-sectional area of the front end portion 221. This suggests that the external form of the present invention is not limited to any specific one.

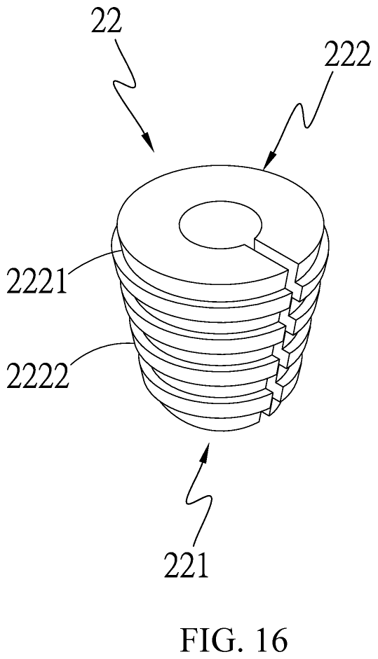

Referring to FIG. 16, in the instant embodiment, an inclined portion 2221 is arranged between the front end portion 221 and the rear end portion 222. The inclined portion 2221 is provided with at least one the rib 2222. This suggests that the fixing member 22 of a detachable fashion may also be provided with the rib 2222 to enhance the fixing effect of the nasogastric tube.

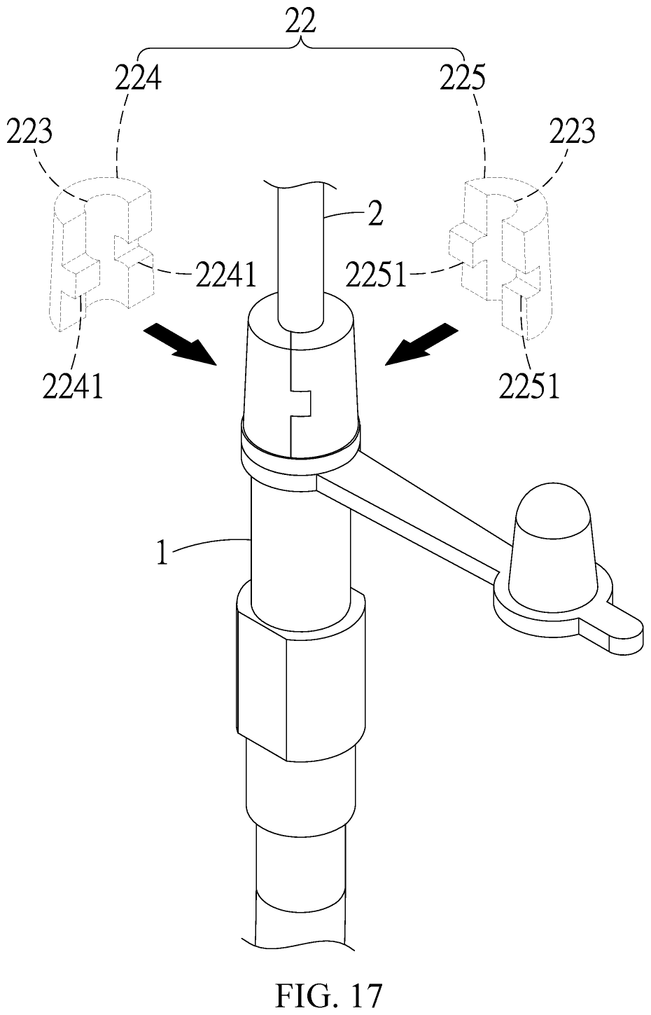

Referring to FIG. 17, the instant embodiment the fixing member 22 is structured in such a way that the fixing member 22 is formed by combining a first fixing member 224 and a second fixing member 225. In other words, the first fixing member 224 includes a first fixing member joining portion 2241, and the second fixing member 225 includes a second fixing member joining portion 2251, and the first fixing member joining portion 2241 and the second fixing member joining portion 2251 are joinable with each other, and the way of joining can be engagement between projections and recesses or tight fitting between projections and recesses. When the first fixing member 224 and the second fixing member 225 are joined to form the fixing member 22, the endoscope 2 can be clamped and thus fixed in the combining portion 223 formed between the first fixing member 224 and the second fixing member 225, to thereby fulfill an advantage of mounting the fixing member 22 on the endoscope 2, and also to achieve an advantage of effectively preventing the endoscope 2 from being excessively inserted into the nasogastric tube 1, and also exhibits an advantage of fast mounting and dismounting.

The present invention provides the following advantages in the use thereof:

By means of the arrangement of the front end portion 221, the inclined portion 2221, and the rear end portion 222 of the fixing member 22, the endoscope 2 is allowed to fast mutually engaging and fixing with the nasogastric tube 1, without using any third-party component, and showing advantages of simple structure and time and cost saving.

By means of the arrangement of the rib 2222, the wall of the nasogastric tube 1 can be further expanded to achieve a better effect of fixing.

The fixing member 22 according to the present invention can be combined with the endoscope 2 in an integrally-formed one-piece structure, or can be combined with an existing endoscope 2 in an externally mountable manner, and as such, as a whole, wide application of use can be realized.

In the case that the fixing member 22 according to the present invention and the endoscope 2 are arranged in a detachable manner, in which an existing endoscope 2 that does not have an effect of fixing can be converted into an endoscope 2 having an effect of fixing, and as a whole, this is convenient and shows better utilization.

I claim:

1. An endoscope fixing structure, mainly comprising:
an endoscope, which is extendable into a nasogastric tube;
an image pickup assembly, which is arranged at an end of the endoscope; and
a fixing member, which is arranged at one side of the endoscope that is opposite to the image pickup assembly, wherein the fixing member comprises a front end portion and a rear end portion having a cross-sectional area greater than the front end portion, so that the fixing member is fixable, by means of the rear end portion, to an opening of the nasogastric tube to constrain movement of the endoscope.

2. The endoscope fixing structure according to claim 1, wherein an inclined portion is arranged between the front end portion and the rear end portion.

3. The endoscope fixing structure according to claim 2, wherein a cross-sectional area of the front end portion is smaller than a cross-sectional area of the opening of the nasogastric tube.

4. The endoscope fixing structure according to claim 3, wherein the cross-sectional area of the rear end portion is greater than the cross-sectional area of the opening.

5. The endoscope fixing structure according to claim 2, wherein at least one rib is formed on the inclined portion.

6. The endoscope fixing structure according to claim 1, wherein the image pickup assembly comprises an image pickup lens, at least one light-emitting element arranged at one side of the image pickup lens, and a signal transmission element arranged at one side of the image pickup lens.

7. The endoscope fixing structure according to claim 6, wherein the signal transmission element comprises one of wired transmission and wireless transmission.

8. The endoscope fixing structure according to claim 6, wherein the signal transmission element is in information connection with a display device.

9. The endoscope fixing structure according to claim 1, wherein the nasogastric tube is formed, in an end, with an end opening to allow the image pickup assembly to pick up an image outside of the nasogastric tube.

10. An endoscope fixing structure, mainly comprising:
a fixing member, which is arranged on an endoscope that is extendable into a nasogastric tub, wherein the fixing member comprises a front end portion and a rear end portion having a cross-sectional area greater than the front end portion, so as to be fixable to an opening of the nasogastric tube to constrain movement of the endoscope; and
a combining portion, which is arranged on the fixing member to receive the endoscope to attach thereto or detach therefrom.

11. The endoscope fixing structure according to claim 10, wherein an inclined portion is arranged between the front end portion and the rear end portion.

12. The endoscope fixing structure according to claim 11, wherein a cross-sectional area of the front end portion is smaller than a cross-sectional area of the opening of the nasogastric tube.

13. The endoscope fixing structure according to claim 12, wherein the cross-sectional area of the rear end portion is greater than the cross-sectional area of the opening.

14. The endoscope fixing structure according to claim 11, wherein at least one rib is formed on the inclined portion.
